(19) United States
(12) Patent Application Publication
(10) Pub. No.: US 2025/0127959 A1
(43) Pub. Date: Apr. 24, 2025

(54) METHODS TO INHIBIT OSTEOPOROSIS THROUGH IMPLANTATION

(71) Applicant: Tarifa Adam, Albany, NY (US)

(72) Inventor: Tarifa Adam, Albany, NY (US)

(21) Appl. No.: 18/493,531

(22) Filed: Oct. 24, 2023

(51) Int. Cl. A61L 27/30; A61L 27/02; A61L 27/54

(52) U.S. Cl. CPC A61L 27/306; A61L 27/025; A61L 27/54; A61L 2300/10; A61L 2300/412; A61L 2430/02

(57) ABSTRACT

In an example, nitrogen is implanted into a bone and heated so as to be incorporated into the bone.

### TECHNICAL FIELD

The present invention relates generally to prevention of osteoporosis, and, in particular embodiments, to methods to inhibit osteoporosis through implantation.

### BACKGROUND

Poor bone density has been one of the primary reasons for the onset of osteoporosis. Therefore, any treatment to improve the density of the bone can help retard or arrest the onset of osteoporosis.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 illustrates a process of implanting nitrogen into a bone structure in accordance with embodiments of the invention;

FIG. 2 illustrates performing a heat treatment to the nitrogen implanted bone in accordance with embodiments of the invention;

FIG. 3 illustrates a bone structure after heat treatment in accordance with embodiments of the invention;

### DETAILED DESCRIPTION OF ILLUSTRATIVE EMBODIMENTS

The present disclosure relates to the field of biomedical implantation and a method for practicing the same. More particularly, the present disclosure relates to a bone whose structure has been modified in order to improve the strength and therefore prevent the progression of osteoporosis.

One possible approach for creating stronger bones is through the implantation of nitrogen and subsequent heat treatment where the nitrogen diffuses towards the surface of the bone. Nitrogen at the surface helps improve the strength of the bone by filling dangling bonds at the bone interface and thereby improving the bone strength. Improved bone strength then helps retard or arrest the onset of osteoporosis in living beings.

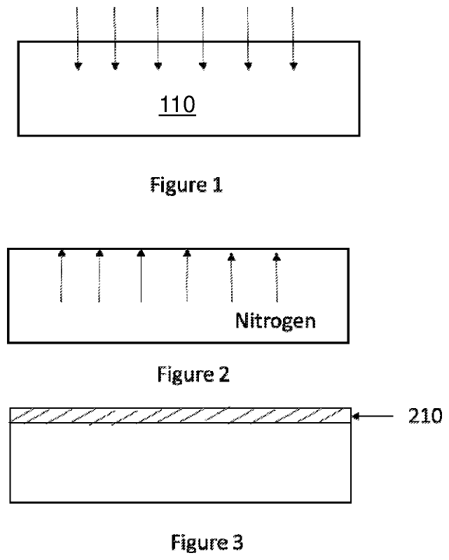

Turning to the drawings, FIG. 1 shows a pictorial representation of nitrogen being implanted into the bone 110. Bone 110 may be in any region of the body of a living being and of any shape or size. Nitrogen is then implanted in to the top of the bone 110.

Any method of implantation can be used. While it is preferred nitrogen to be selectively implanted into the target bone through the use of masking techniques, it is not a requirement for this method. The recommended dose can be in the range of up to 1×10¹⁵/cm².

In FIG. 2, heat is applied in the range of up to 50° C. and the implanted nitrogen is then brought to the surface of the bone (110) through diffusion. Any process of heat treatment will suffice.

FIG. 3 depicts the structure of the bone subsequent to the method described in the schematic of FIG. 2. After the diffusion of nitrogen towards the surface, the layer 210 is formed. The foregoing description of the disclosure illustrates and describes the present disclosure. Additionally, the disclosure shows and describes only the preferred embodiments but, as mentioned above, it is to be understood that the disclosure is capable of use in various other combinations, modifications, and environments and is capable of changes or modifications within the scope of the concept as expressed herein, commensurate with the above teachings and/or the skill or knowledge of the relevant art.

The embodiments described hereinabove are further intended to explain best modes known of practicing it and to enable others skilled in the art to utilize the disclosure in such, or other, embodiments and with the various modifications required by the particular applications or uses.

Accordingly, the description is not intended to limit it to the form disclosed herein. Also, it is intended that the appended claims be construed to include alternative embodiments.

What is claimed is:

1. A structure:
a bone comprising an inner core and a peripheral region;
the peripheral region incorporating nitrogen atoms.

2. A method comprising:
implanting nitrogen into a bone; and
heating the bone with the implanted nitrogen.

3. The method of claim 2, wherein the implantation is performed in a dose range up to 1×10¹⁵/cm².

4. The method of claim 2, wherein the heating is performed in a temperature range of up to 50° C.

5. The method of claim 2, wherein implanting the nitrogen comprises selectively implanting nitrogen into a portion of the bone.

6. The method of claim 5, wherein the portion of the bone is a periosteum.
